(19) United States
(12) Patent Application Publication
(10) Pub. No.: US 2026/0053647 A1
(43) Pub. Date: Feb. 26, 2026

(54) MODULAR PROSTHETIC DEVICE

(71) Applicant: Phoenix Biomedical LLC, Chesterfield, MO (US)

(72) Inventors: Lili Hostetler, Chesterfield, MO (US); Kaitlyn Thornton, St. Louis, MO (US); Jenna (Logan) Rogge, Lincoln, NE (US)

(21) Appl. No.: 19/309,071

(22) Filed: Aug. 25, 2025

Related U.S. Application Data
(60) Provisional application No. 63/686,911, filed on Aug. 26, 2024.
(60) Provisional application No. 63/742,464, filed on Jan. 7, 2025.

(51) Int. Cl. A61F 2/78; A61F 2/50; A61F 2/60; A61F 2/66

(52) U.S. Cl. CPC A61F 2/7812; A61F 2002/5083; A61F 2002/607; A61F 2002/6642; A61F 2002/6657

(57) ABSTRACT

A modular prosthesis including a socket assembly having an outer socket and an inner socket coupled to the outer socket. The modular prosthesis includes a base configured to contact a ground surface, and a pylon disposed between the socket assembly and the base. The pylon defines a longitudinal axis. The pylon has a first end and a second end opposite from the first end along the longitudinal axis. The first end is coupled to the socket assembly and the second end is coupled to the base. The modular prosthesis includes a first locking mechanism disposed between the socket assembly and the pylon. The modular prosthesis includes a second locking mechanism disposed between the pylon and the base.

The application claims priority to U.S. Provisional Patent Application No. 63/742,464, filed Jan. 7, 2025, and U.S. Provisional Patent Application No. 63/686,911, filed Aug. 26, 2024, the entire contents of all of which are incorporated by reference herein.

### FIELD OF THE DISCLOSURE

The present disclosure relates to prosthetic devices, and more particularly to a modular prosthesis that is selectively adjusted to accommodate and support a user. Also, the present disclosure relates to prosthetic devices, and more particularly to maintenance of prosthetic devices.

### BACKGROUND OF THE DISCLOSURE

Prostheses act as an artificial replacement for a missing body part. A person may have been born without the body part, or the body part may have been amputated due to an illness, accident, infection, etc. Regardless of the reason, prostheses provide a user with increased mobility, independence, and comfort, and improve overall quality of life. There are various types of prostheses that accommodate or adapt to different areas of the body. In some cases, prostheses may be passive, and they may serve solely aesthetic purposes. In other cases, prostheses may have active functioning, where the prostheses provide movement to a user. While there are numerous prostheses that are each intended to address certain conditions and needs, there are four common types of prostheses: transradial, transfemoral, transtibial, and transhumeral. The transradial and transhumeral prostheses are artificial body parts of the upper extremity, while the transfemoral and transtibial prostheses are artificial body parts of the lower extremity.

Because a prosthesis is to serve a user in all their physical and life endeavors, it is pertinent that a prosthesis be durable, comfortable, and unique to the user. For obtaining a prosthesis, a user may visit a prosthetist to evaluate the user's needs and begin the process of fitting the user for a prosthesis (e.g., take measurements relating to the designated area for the prosthesis, create a cast, and develop a plaster impression, amongst others). However, the pains of the process for fitting and receiving a prosthesis do not end with receiving the first prosthesis. Due to bodily changes (for example, growth, muscle hypertrophy, muscle atrophy, etc.) and other factors, a user may have to visit a prosthetist to alter their prosthesis to meet their new needs. For example, an adolescent user may need to visit their prosthetist multiple times through adulthood to align with growth spurts. As another example, a user's muscles may atrophy, and the prosthesis may need to be adjusted to accommodate the respective body area and new muscle mass. For some, visiting the prosthetist may be cumbersome and, in instances where a prosthesis may need multiple minor adjustments, a user may journey to the prosthetist multiple times. Moreover, obtaining a prosthesis and having multiple prosthetist appointments may be an expense inaccessible to those of lower incomes. As such, there is a long-felt but unresolved need for a prosthesis that is user-friendly and user-customizable, such that the user may adjust the prosthesis to meet their daily and long-term needs while still being customizable, durable, affordable, comfortable, and adaptable. Prosthetic devices may require an initial fitting, subsequent fittings, replacements, and maintenance over the lifetime of the prosthetic device.

### SUMMARY OF THE DISCLOSURE

The present disclosure provides, in one aspect, a modular prosthesis including a socket assembly having an outer socket and an inner socket coupled to the outer socket. The modular prosthesis includes a base configured to contact a ground surface, and a pylon disposed between the socket assembly and the base. The pylon defines a longitudinal axis. The pylon has a first end and a second end opposite from the first end along the longitudinal axis. The first end is coupled to the socket assembly and the second end is coupled to the base. The modular prosthesis includes a first locking mechanism disposed between the socket assembly and the pylon. The modular prosthesis includes a second locking mechanism disposed between the pylon and the base.

The present disclosure provides, in another aspect, a method of fitting and assembling a modular prosthesis. The method includes heating an inner socket with a heat source, conforming a residual limb to the inner socket, coupling, with a shuttle lock, the inner socket to an outer socket, coupling, with fasteners, a first connector to the outer socket, coupling, with a threaded connection, a first end of a first pylon to the first connector of a first locking mechanism, coupling, with a threaded connection, a second end of the first pylon to a second connector of a second locking mechanism, and coupling, with fasteners, the second connector to a base.

Other features and aspects of the disclosure will become apparent by consideration of the following detailed description and accompanying drawings.

### BRIEF DESCRIPTION OF THE DRAWINGS

FIG. 1 is a front view of a modular prosthesis according to the present disclosure.

FIG. 2 is a front view of a socket assembly of the modular prosthesis of FIG. 1.

FIG. 3 is an isometric view of the socket assembly of FIG. 2.

FIG. 4 is a rear view of the socket assembly of FIG. 2.

FIG. 5 is an isometric view of a pylon of the modular prosthesis of FIG. 1.

FIG. 6 is an isometric view of a base of the modular prosthesis of FIG. 1.

FIG. 7 is a top view of the base of FIG. 6.

FIG. 8 is a side view of the base of FIG. 6.

FIG. 9 is an isometric view of a first connector of the modular prosthesis of FIG. 1.

FIG. 10 is a top view of the first connector of FIG. 9.

FIG. 11 is a bottom view of the first connector of FIG. 9.

FIG. 12 is a front view of the socket assembly and the first connector of the modular prosthesis of FIG. 1 coupled together.

FIG. 13 is an isometric view of a second connector of the modular prosthesis of FIG. 1.

FIG. 14 is a top view of the second connector of FIG. 13.

FIG. 15 is a bottom view of the second connector of FIG. 13.

FIG. 16 is a front view of a socket assembly compatible with the modular prosthesis of FIG. 1.

FIG. 17 is an isometric view of an inner socket the socket assembly of FIG. 16.

FIG. 18 is a cross-sectional view of the inner socket taken along line 18-18 in FIG. 17.

FIG. 19 is an isometric view of an outer socket of the socket assembly of FIG. 16.

FIG. 20 is a rear isometric view of the outer socket of FIG. 19.

FIG. 21 is a cross-sectional view of the outer socket taken along line 21-21 in FIG. 20.

FIG. 22 is an isometric view of a shuttle lock mechanism.

FIG. 23 is an exploded view of a modular prosthesis according to another embodiment of the present disclosure.

FIG. 24 is an isometric view of a pylon cover for the modular prosthesis of FIG. 1.

FIG. 25 is an isometric view of a ratchet strap for use with the modular prosthesis of FIG. 1.

FIG. 26 is a side view of the ratchet strap of FIG. 24.

FIG. 27 is a block diagram of maintenance repair of a prosthetic.

FIG. 28 is a block diagram of an initial fitting of a prosthetic.

FIG. 29 is a block diagram of maintenance repair of a prosthetic, according to an embodiment of the disclosure.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting.

### DETAILED DESCRIPTION

It is to be understood that the use of “femur,” “femoral shaft,” “thigh,” and variations thereof is indicative of the area in human anatomy that extends between a person's hip and knec joints. The use of “leg,” “tibia,” “fibula,” and variations thereof is indicative of the area in human anatomy that extends between the knee and ankle joints. The use of “pedal” and “foot” is indicative of the area beyond the ankle joint and inclusive of the lower phalanges. The use of “lower extremity” includes combinations of the femur, the leg, and the foot and the joints located therebetween such as the knee and ankle. Moreover, the use of “residual limb” is indicative of the remaining portion of a user's body part (for example, of a femur or leg).

The present disclosure is directed to a modular prosthesis 100 that may be fitted, adjusted, modulated, and customized to meet the needs of a user. Unlike current prostheses, the modular prosthesis 100 described herein comprises adjustable prosthetic components. Generally, the modular prosthesis 100 is an active functioning, artificial body part designed to provide and mimic the functioning of the missing limb. In some constructions, the modular prosthesis 100 is a lower extremity prosthesis designed to facilitate and accommodate a user's gait. In such instances, the modular prosthesis 100 is disposed about a user's residual limb (for example, the femur) and extend downwards towards the ground to replace the user's leg, ankle, and foot. The modular prosthesis 100 is manufactured, heat-molded, and/or 3D printed from a lightweight, durable, material. In some constructions, the modular prosthesis 100 includes materials such as a plastic, aluminum, titanium, and silicone. In some constructions, the modular prosthesis 100 is 3D printed and includes materials such as UV-curable elastomers, plastics, and/or nylon. In yet other instances, the modular prosthesis 100 includes biocompatible materials.

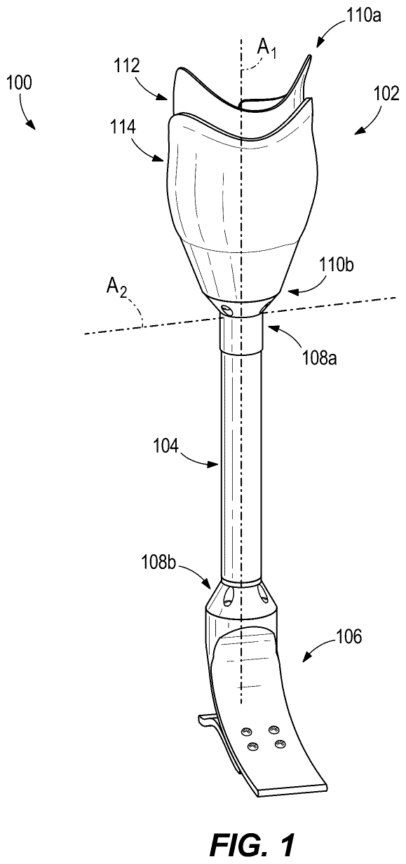

FIG. 1 illustrates an embodiment of a lower-extremity modular prosthesis 100. As referred to herein, it is to be understood that a longitudinal axis A1 is an imaginary or hypothetical line used to describe the orientation, positioning, location, and the like of varying components in relation to each other. It is also to be understood that the longitudinal axis A1 corresponds to a sagittal plane of common anatomical nomenclature. As used herein, the longitudinal axis A1 is used to describe modular prosthesis features disposed to the left and right of the longitudinal axis A1 and/or modular prosthesis features disposed towards a top (i.e., upper portion) or a bottom (i.e., lower portion) with respect to the longitudinal axis A1. Additionally, symmetry about the longitudinal axis A1 is used to describe two sections of one component having a substantial mirror likeness, or a balanced and proportionate similarity, with respect to the axis (i.e., a vertical symmetry when reflected over the longitudinal axis A1). Further, the longitudinal axis A1 describes a length of a prosthetic component and an increasing or decreasing length of said prosthetic component.

Similar to the longitudinal axis A1, it is to be understood that a lateral axis A2 is an imaginary or hypothetical line referred to herein to describe the orientation, positioning, location, and the like of varying components in relation to each other. It is also to be understood that the lateral axis A2 corresponds to a transverse plane of common anatomical nomenclature. As used herein, the lateral axis A2 is used to describe modular prosthesis features disposed above and below the lateral axis A2 and/or modular prosthesis features disposed towards the left or right with respect to the lateral axis A2. Additionally, symmetry about the lateral axis A2 is used to describe two sections of one component having a substantial mirror likeness, or a balanced and proportionate similarity, with respect to the axis (i.e., a horizontal symmetry when reflected over the lateral axis A2). In some constructions, the lateral axis A2 describes a width of a component and an increasing or decreasing width of said component.

Continuing reference to FIG. 1, the modular prosthesis 100 includes a socket assembly 102, a pylon 104, and a base 106. The socket assembly 102 is coupled to the pylon 104 with a first locking mechanism 108a. The pylon 104 and the base 106 are coupled to one another with a second locking mechanism 108b. Generally, the modular prosthesis 100 is adjustable (e.g., in length, width, and/or circumference) due to the scalability of the socket assembly 102, the pylon 104, and the base 106 such that the modular prosthesis 100 accommodates a user's changing growth, weight, muscle density, and/or age. In some constructions, although not illustrated, the prosthetic components (e.g., socket assembly 102, pylon 104, and base 106) each include telescoping members. The telescoping members permit adjustment of a length and width of the prosthetic component. For example, the pylon 104 includes two telescoping members that rotate, slide, and/or twist, with respect to each other, to adjust a length of the pylon 104. In another example, the length of the socket assembly 102 is adjusted to a desired size by vertically pulling or pushing the socket assembly 102, with respect to the longitudinal axis A1. In some constructions, the modular prosthesis 100 is adjusted to support bodily changes for the user's current stage in life (e.g., toddlerhood, preschool years, early school years, adolescence, young adulthood, middle adulthood, and late adulthood).

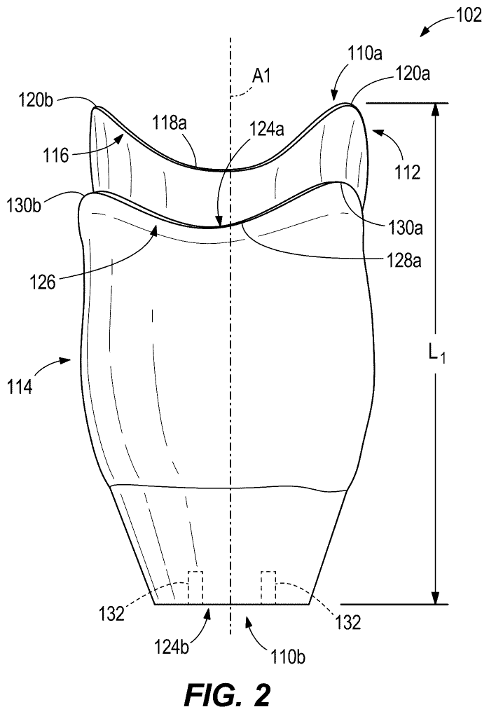

FIGS. 2-4 illustrate the socket assembly 102 of the modular prosthesis 100. The socket assembly 102 is designed to substantially (1) receive a user's residual limb, (2) protect the residual limb, and (3) accommodate the needs of the user by adjusting the size and shape of the socket to compliment the fluctuations of the residual limb's size and shape. The socket assembly 102 includes a first socket end 110a and a second socket end 110b, where the distance between the first and second socket ends 110a, 110b is a socket length L1 measured along the axis A1. In some constructions, the socket assembly 102 includes an inner socket 112 and an outer socket 114. In some constructions, the inner socket 112 and the outer socket 114 are each vertically symmetrical. Moreover, the inner socket 112 is most proximal to a user's skin. In one example, when the socket assembly 102 is worn, the inner socket 112 establishes direct contact with the user. Preferably, the inner socket 112 is conformable to the shape of the user's residual limb.

With continued reference to FIGS. 2-4, the inner socket 112 includes an upper periphery 116, relative to the first socket end 110a, that has a combination of dips and peaks along the front, left, right, and rear sides of the inner socket 112. In some constructions, the dips are points about the upper periphery 116 that extend downwards with respect to the longitudinal axis A1 (e.g., toward the pylon 104). In such instances, the dips are the lowest points of the upper periphery 116. In contrast, the peaks are points about the upper periphery 116 that extend upwards with respect to the longitudinal axis A1 (e.g., away from the pylon 104). The peaks are the highest points about the upper periphery 116. The front of the upper periphery 116 includes a first dip 118a, and the rear of the upper periphery 116 includes a second dip 118b. Moreover, the upper periphery 116 includes a first peak 120a that is disposed about the left side of the upper periphery 116, and a second peak 120b that is disposed about the right side of the upper periphery 116. In some constructions, the second dip 118b extends lower than the first dip 118a. In some constructions, the first and second dips 118a, 118b are about the same depth. In some constructions, the first dip 118a, the second dip 118b, the first peak 120a, and the second peak 120b, are substantially the same height. In the illustrated construction, the second dip 118b is separated by a slit 122. The slit 122 extends in a direction parallel to the longitudinal axis A1 and toward the second socket end 110b. Moreover, the upper periphery 116 defines a socket opening 123 that is designed to receive the user's residual limb.

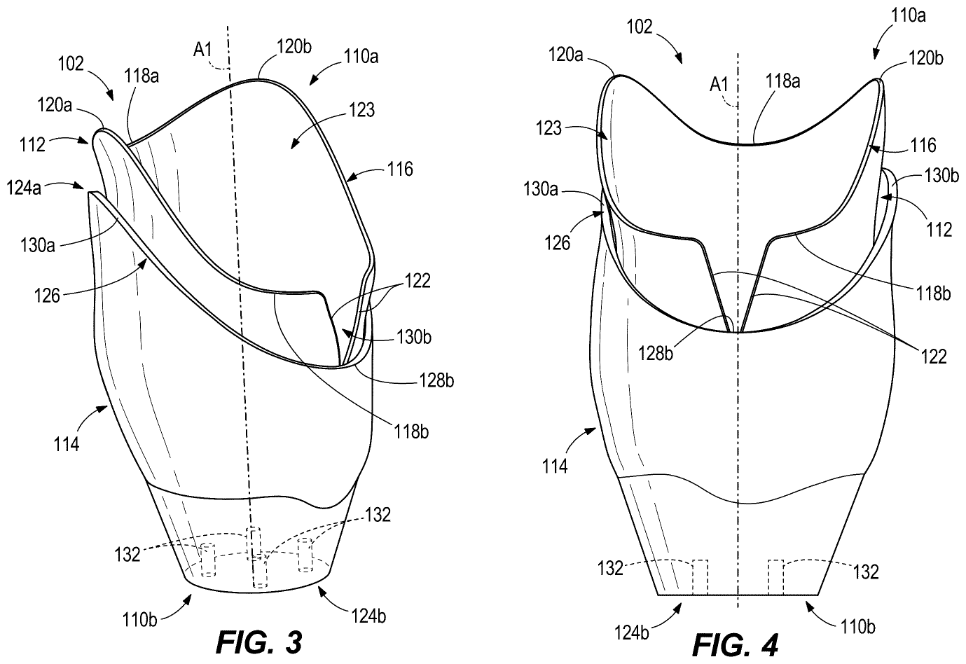

Surrounding (i.e., encasing) the inner socket 112 is the outer socket 114. Generally, the outer socket 114 is designed as a “shell” around the inner socket 112. Whereas the inner socket 112 is the most proximal to the user, the outer socket 114 is relatively more distal. In some constructions, the inner and outer sockets 112, 114 are integrally formed (i.e., uniformly manufactured, created, 3D printed, and the like). In some constructions, the inner and outer sockets 112, 114 are independently manufactured and selectively assembled or removed from each other. As best illustrated in FIG. 3, the outer socket 114 is separated by a distance from the inner socket 112. In some constructions, the outer socket 114 is about 50% of the socket length L1, leaving about 50% of the inner socket 112 exposed. In some constructions, the outer socket 114 is greater than 50% of the socket length L1. The outer socket 114 defines a larger width than the inner socket 112 since the outer socket 114 encases the inner socket 112. In some constructions, between an upper end 124a and a lower end 124b of the outer socket 114, the outer socket 114 defines a gradually changing width. In some constructions, the width of the outer socket 114 is substantially constant. The front and rear surfaces of the outer socket 114 defines a shape that is relatively flatter, while the left and right surfaces of the outer socket 114 are relatively more rounded or concave. In some constructions, the outer socket 114 has a substantially more rounded shape than the inner socket 112.

About the upper end 124a of the outer socket 114, the outer socket 114 defines an upper periphery 126 having a plurality of dips and peaks along the front, left, right, and rear sides. In some constructions, the dips are points about the upper periphery 126 that extend downwards with respect to the longitudinal axis A1 (e.g., toward the pylon 104). In such instances, the dips are the lowest points of the upper periphery 126. In contrast, the peaks are points about the upper periphery 126 that extend upwards with respect to the longitudinal axis A1 (e.g., away from the pylon 104). The peaks are the highest points about the upper periphery 126. For example, as best illustrated in FIGS. 3 and 4, the front of the upper periphery 126 includes a first dip 128a, and the rear of the upper periphery 126 includes a second dip 128b. Moreover, the upper periphery 126 includes a first peak 130a that is disposed about the left side of the upper periphery 126, and a second peak 130b that is disposed about the right side of the upper periphery 126. In some constructions, the second dip 128b extends lower than the first dip 128a. In some constructions, the first and second dips 128a, 128b are about the same depth. In some constructions, the first dip 128a, the second dip 128b, the first peak 130a, and the second peak 130b, are substantially the same height. In the illustrated construction, the dips 118a-b of the inner socket 112 radially align with the dips 128a-b of the outer socket 114 relative to the longitudinal axis A1. In the illustrated construction, the peaks 120a-b of the inner socket 112 radially align with the peaks 130a-b of the outer socket 114 relative to the longitudinal axis A1. In the illustrated construction, the outer socket 114 includes apertures 132 disposed on the lower end 124b. The apertures 132 receive fasters to couple the outer socket 114 to the pylon 104 and/or the first locking mechanism 108a. The connection between the outer socket 114 and the pylon 104 and/or the first locking mechanism 108a will be discussed later in the application.

In some constructions, the inner and outer sockets 112, 114 are formed by heat molding and/or includes heat moldable materials. For example, the inner socket 112 is 3D-printed using a heat-moldable plastic material or a combination of heat-moldable materials. In this example, the inner socket 112 was initially printed to conform to the current size and shape of the user's residual limb. However, as the user's needs change, the inner socket 112 is re-heated via a microwave, oven, and the like, to adjust the shape and size of the inner socket 112 thus enabling the user to selectively modify the fit between the inner socket 112 and the residual limb. Similarly, the outer socket 114 is printed from a heat-moldable material. The outer socket 114 is re-heated via similar means to adjust the shape and size of the outer socket 114 to accommodate the changing size of the inner socket 112.

In some constructions, the socket length L1 is adjustable. For example, a user may manually manipulate the socket assembly 102 by pulling the first socket end 110a and the second socket end 110b in opposite longitudinal directions, relative to the longitudinal axis A1, thus increasing the socket length L1. In another example, the user may manually manipulate the socket assembly 102 by pushing the first socket end 110a and the second socket end 110b towards each other, thus decreasing the socket length L1. The adjusted socket length L1 is secured by rotating, twisting, or using a fastener (for example, lock and pins, levers, latches, and the like). In some constructions, although not illustrated, the socket assembly 102 includes an adjustable width (e.g., in a direction perpendicular to the longitudinal axis A1). As discussed previously, the socket assembly 102 is heated to adjust the fit. In some constructions, the socket assembly 102 is heated to adjust the width. In some constructions, a user may laterally pull apart the left and right sides of the socket assembly 102, with respect to the lateral axis A2. Alternatively, to decrease the width of the socket assembly 102, the user may laterally push the left and right sides of the socket assembly 102 together, with respect to the lateral axis A2.

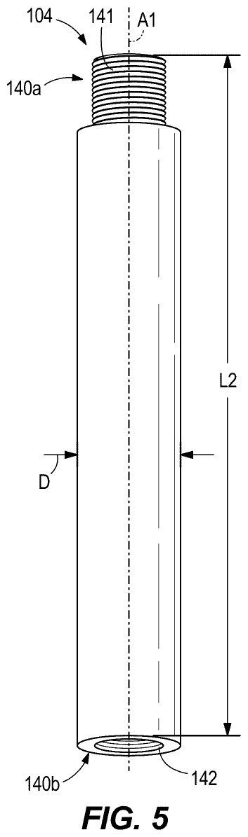

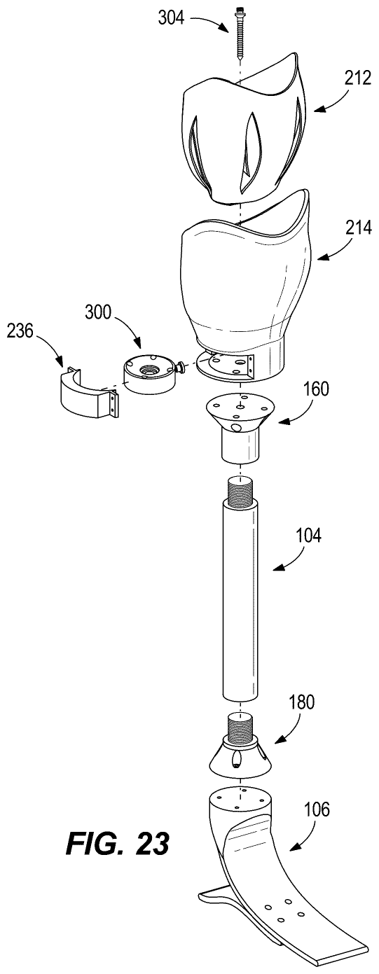

FIG. 5 illustrates the pylon 104 defining a cylindrical shape. The pylon 104 defines a diameter D. For a human lower extremity, the pylon 104 is aesthetically and/or functionally similar to the user's respective lower anatomy. In other constructions, a cover is attached to the pylon 104 to aesthetically match the user's respective lower anatomy (FIG. 23). For example, the pylon 104 functions similarly to a human leg (e.g., a tibia and/or a fibula) such that the pylon 104 is configured to provide weight bearing capabilities. In another example, the pylon 104 provides functionality similar to a human thigh (e.g., a femur). In yet another example, the pylon 104 provides functionality of both a human thigh and leg. The pylon 104 includes a first pylon end 140a and an opposing second pylon end 140b. In some constructions, the first pylon end 140a is proximal to the socket assembly 102 and the second pylon end 140b is proximal to the base 106. In the illustrated construction, the first pylon end 140a includes a protrusion with threads 141 (e.g., a male end). In the illustrated construction, the second pylon end 140b includes a receptacle 142 that is threaded (e.g., a female end).

In the illustrated construction, the pylon 104 defines a pylon length L2 along the longitudinal axis A1. In the illustrated construction, the pylon length L2 is fixed. In other words, the pylon length L2 is not adjustable by manipulating the pylon 104.

In other constructions, the pylon 104 is a telescoping member. That is, the pylon 104 is capable of length adjustment along the longitudinal axis A1 (e.g., increasing or decreasing the pylon length L2 along the axis A1). For example, the first and second pylon ends 140a, 140b are vertically pulled apart, with respect to the longitudinal axis A1, to increase the pylon length L2. Alternatively, the first and second pylon ends 140a, 140b are vertically pushed together, with respect to the longitudinal axis A1, to decrease the pylon length L2. In some constructions, although not shown, the pylon 104 includes at least two telescoping members. The telescoping members of the pylon 104 may slide, rotate, or twist away or towards each other and, upon reaching a desired length, the telescoping members are further manipulated to be secured in place. Alternatively, the pylon length L2 is secured by another fastener. In some constructions, although not illustrated, the pylon 104 is configured to receive pylon extension pieces. The pylon extension pieces may couple to an end of the pylon 104 and adjacent pylon extension pieces to extend the pylon length L2.

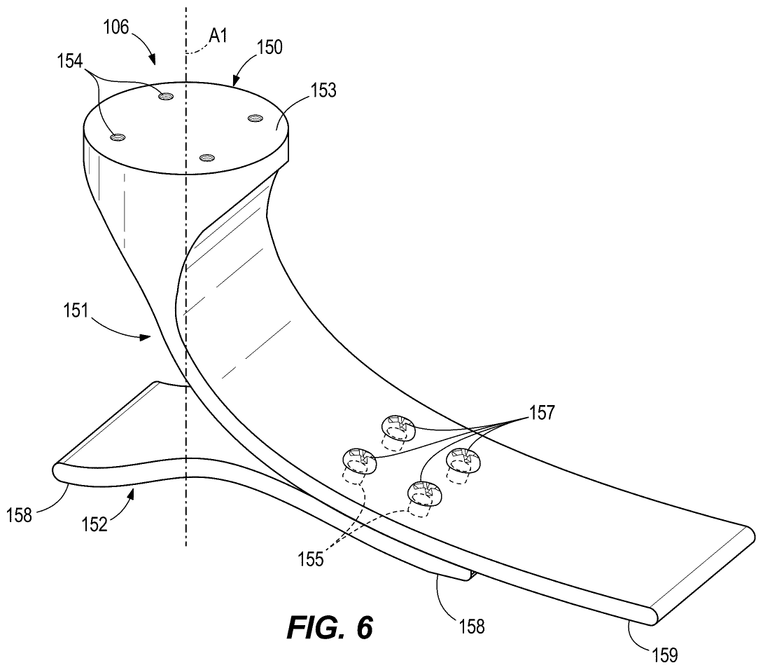

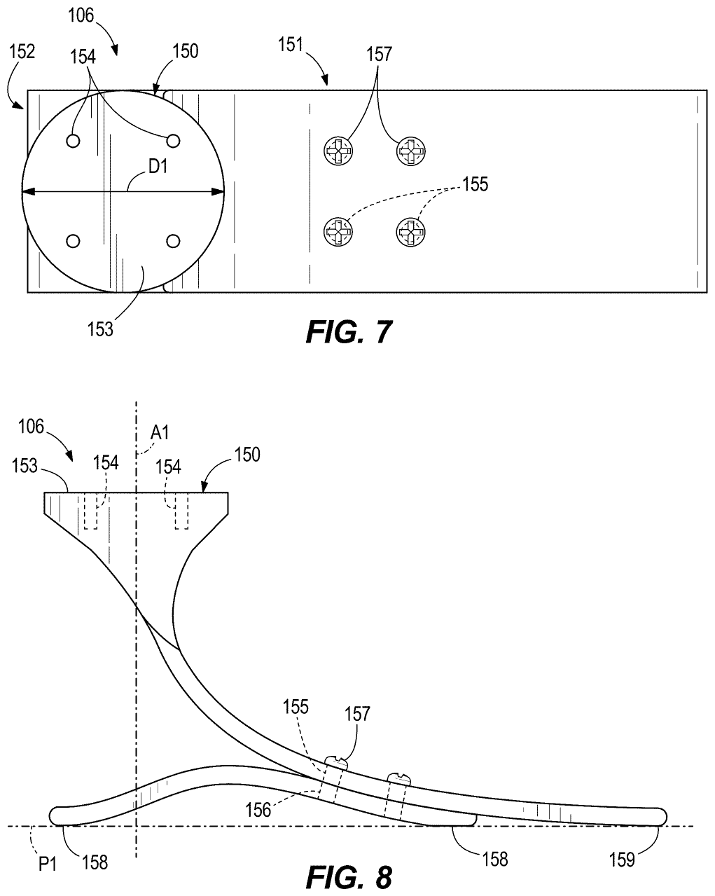

Referring to FIGS. 6-8 the base 106 includes a platform 150, a blade 151 extending from the platform 150, and a heel 152 coupled to the blade 151. In the illustrated construction, the platform 150 and the blade 151 are integrally formed (e.g., a monolithic and/or a single piece construction including the platform 150 and the blade 151). In some constructions, the platform 150 and the blade 151 are independently formed and later assembled. In the illustrated construction, the blade 151 and the heel 152 are independent components (e.g., the blade 151 and the heel 152 are separately formed and are later assembled). In some constructions, the blade 151 and the heel 152 are integrally formed (e.g., a monolithic and/or a single piece construction including the blade 151 and the heel 152). When the modular prosthesis 100 is worn, the platform 150, the blade 151, and the heel 152 are substantially aesthetically and/or functionally similar to a human ankle and foot, respectively. The base 106, in combination with the socket assembly 102 and the pylon 104, is durable enough to withstand the weight of a user and facilitate balance.

In the illustrated construction, the platform 150 defines a substantially circular or rounded shape. Alternatively, the platform 150 defines an angular shape comprising rounded and rectilinear lines. The platform 150 includes a top face 153 that engages with and couples to the second pylon end 140b or with the second locking mechanism 108b by rotating, twisting, sliding, or another fastening mechanism. In one example, the pylon 104 couples to at least a portion of the top face 153. In some constructions, the platform 150 includes threads on the outer surface that engage with the threads of the pylon 104. In some constructions, the platform 150 includes a plurality of apertures 154. The plurality of apertures 154 are configured to facilitate coupling the pylon 104 and the base 106. For example, the platform 150 includes four apertures 154 that extend through the platform 150 (i.e., the apertures 154 are through holes). In the illustrated construction, the apertures 154 are equally spaced about the longitudinal axis A1. Specifically, the apertures 154 are spaced apart by 90 degrees about the longitudinal axis A1. In the illustrated construction, the apertures 154 are threaded such that the apertures 154 are configured to receive a threaded member. In some constructions, the pylon 104 includes four pegs of a complimentary shape for each of the four apertures 154. Continuing this example, each peg is designed to engage one of the four apertures 154 (i.e., each peg is received by one of the four apertures 154), thus securing the pylon 104 to the base 106.

With continued refence to FIGS. 6-8, the blade 151 defines a substantially “hooked” or “C” shape. After curving downwards, the blade 151 laterally extends away from the platform 150. That is, the blade 151 extends radially away from the longitudinal axis A1 and axially along the longitudinal axis A1. The blade 151 includes a plurality of apertures 155 that extend through the blade 151 (e.g., the apertures 155 are through holes). In the illustrated construction, the plurality of apertures 155 include four apertures.

With refence to FIGS. 6 and 8, the heel 152 defines a gradual inverted U-shape. The heel 152 includes apertures 156 that extend through the heel 152. The apertures 156 align with the apertures 155 of the blade 151. In the illustrated construction, the plurality of apertures 156 include four apertures.

To couple the heel 152 to the blade 151, the apertures 155, 156 are aligned and a fastener of a plurality of fasteners 157 pass through the apertures 155, 156. In the illustrated constructions, the apertures 155, 156 include threads that correspond to threads of the fasteners 157. In some constructions, the base 106 includes a corresponding locking element (e.g., a nut) to lock the plurality of fasteners 157 to the apertures 155, 156 such that the blade 151 and the heel 152 are coupled together. In other constructions, the fasteners 157 are self-locking. In the illustrated construction, the fasteners 157 are pan head screws. 0.25-20×0.5 inches. As shown in FIG. 8, a plane P1 illustrates the ground or a surface that engages the base 106. Specifically, the heel 152 includes two opposite ends 158 that engage the plane P1 and the blade 151 includes an end 159 that engages the plane P1. In other words, there are three points (e.g., the ends 158 and the end 159) on the base 106 that have the same axial location along the longitudinal axis A1 such that the plane P1 is engaged.

Referring briefly back to FIG. 1, each of the components (e.g., the socket assembly 102, pylon 104, and base 106) couple to an adjacent component via the first and second locking mechanisms 108a, 108b. Specifically, the first pylon end 140a includes a first locking mechanism 108a and the second pylon end 140b includes a second locking mechanism 108b. Each of the locking mechanisms 108a, 108b are connectors that mate or otherwise engage with the adjacent components. In some constructions, the locking mechanisms 108a, 108b include one or more levers, pins, pegs, hook and loop fasteners, screws, and the like to securely couple the prosthetic components. In other examples, the locking mechanisms 108a, 108b are a twist-snap lock, a bayonet mount, and the like.

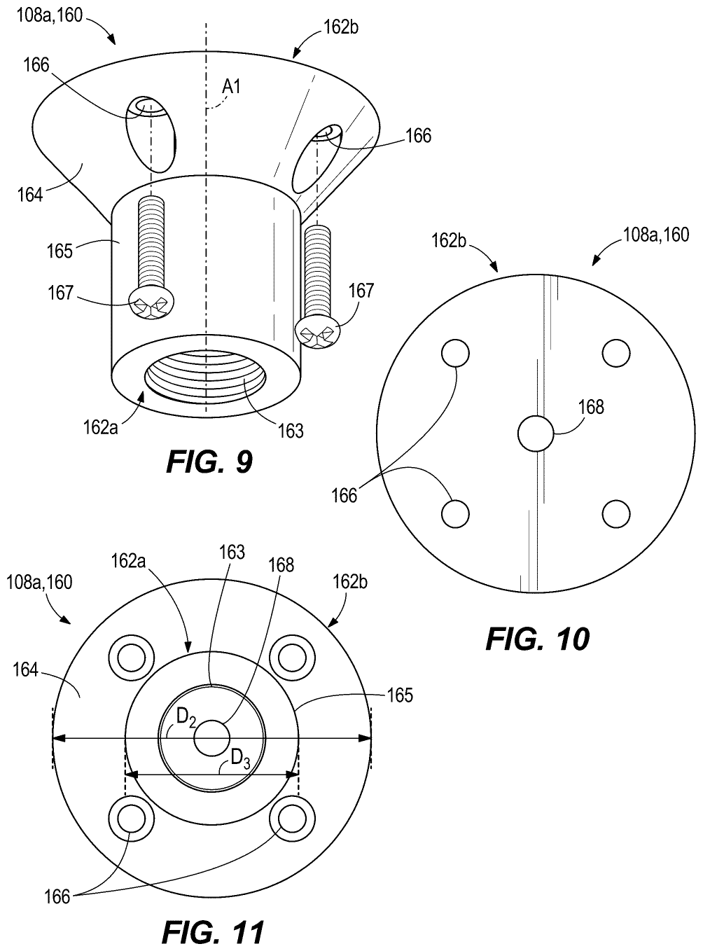

FIGS. 9-11 illustrate the first locking mechanism 108a having a first connector 160 that connects with the first pylon end 140a. The first connector 160 defines a substantially cylindrical shape with a circular cross-section. In some constructions, the first connector 160 is formed integrally with the socket assembly 102, pylon 104, and/or base 106. In the illustrated construction, the first connector 160 is independently formed and later assembled with the modular prosthesis 100. The first connector 160 includes a first end 162a and a second end 162b that is disposed on an opposite end of the first connector 160 relative to the first end 162a along the longitudinal axis A1. The first end 162a includes a receptacle 163 (e.g., a female end) configured to receive the first pylon end 140a. In the illustrated construction, the receptacle 163 is threaded to correspond with the threads on the first pylon end 140a. In other constructions, the receptacle 163 includes corresponding geometry to engage the pylon 104 (e.g., bayonet connection, magnets, ball detent).

With continued reference to FIGS. 9-11, the second end 162b defines a second diameter D2 and the first end 162a defines a third diameter D3. The second diameter D2 is larger than the third diameter D3. The diameter D3 is approximately the same magnitude as the diameter D. As shown in FIG. 9, the second end 162b includes a head 164 that tapers outward from an outer surface 165 of the first connector 160. As shown in FIG. 10, the second end 162b includes apertures 166 configured to align with the apertures 132 of the outer socket 114. In other words, the apertures 166 are disposed at common radial locations and circumferential locations relative to the longitudinal axis A1 such that apertures 132, 166 align. In the illustrated construction, the apertures 166 extend through the head 164 (e.g., the apertures 166 are through holes). In the illustrated construction, the apertures 166 are equally spaced about the longitudinal axis A1. Specifically, the apertures 166 are spaced apart by 90 degrees about the longitudinal axis A1. The modular prosthesis 100 includes fasteners 167 that couple the outer socket 114 to the pylon 104. Specifically, the fasteners 167 extend through the apertures 132, 166. In some constructions, the modular prosthesis 100 includes a locking element (e.g., a nut) to lock the fasteners 167 to the apertures 132, 166 such that the outer socket 114 is coupled to the pylon 104. In other constructions, the fasteners 167 are self-locking such that the outer socket 114 is coupled to the pylon 104. In the illustrated construction, the fasteners 167 are socket head screws M6×1.00P×30 mm. The second end 162b includes an aperture 168 disposed centrally on the head 164. In other words, the aperture 168 is centered with respect to the longitudinal axis A1.

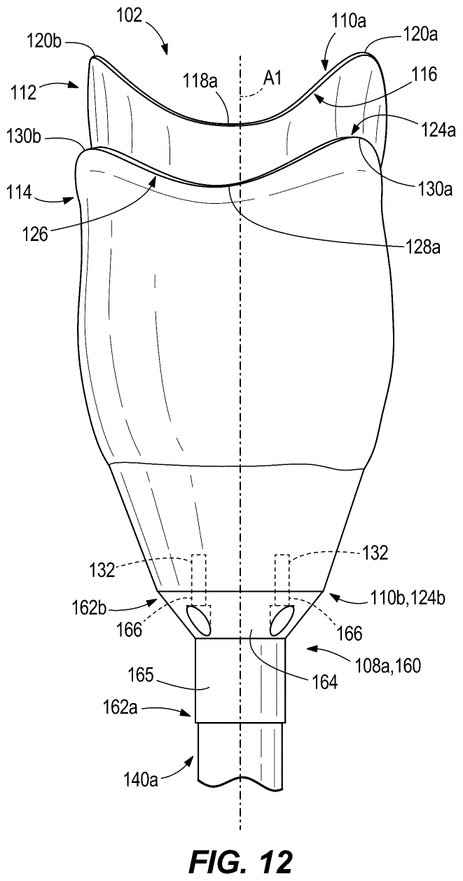

FIG. 12 illustrates the first connector 160 coupled to the pylon 104. The pylon 104 is coupled to the socket assembly 102 about the second socket end 110b such that that the second end 162b is flush against the lower end 124b of the outer socket 114. That is, the socket assembly 102 and the first connector 160 do not axially overlap along the axis A1.

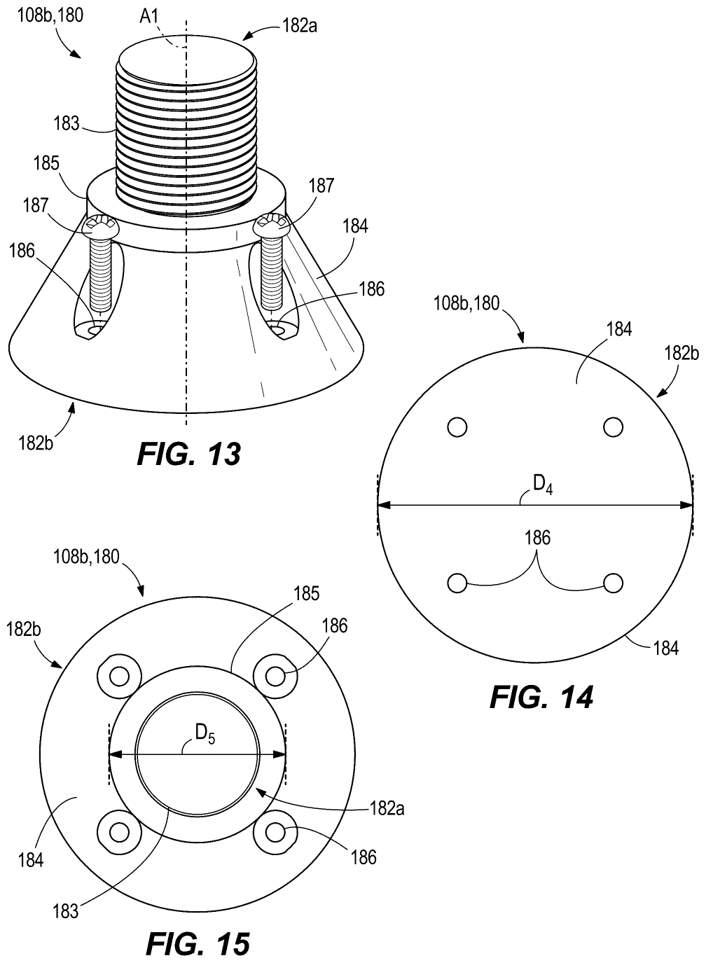

FIGS. 13-15 illustrate the second locking mechanism 108b having a second connector 180 that connects with the second pylon end 140b. The second connector 180 defines a substantially cylindrical shape with a circular cross-section. In some constructions, the second connector 180 is formed integrally with the socket assembly 102, pylon 104, and/or base 106. In some constructions, the second connector 180 is independently formed and later assembled with the modular prosthesis 100. The second connector 180 includes a first end 182a (e.g., male end) and a second end 182b that is disposed on an opposite end of the second connector 180 relative to the first end 182a along the longitudinal axis A1. The first end 182a includes threads 183 that are configured to engage with the threads of the receptacle 142 of the pylon 104. In other constructions, the threads 183 are replaced with other corresponding geometry to engage the pylon 104 (e.g., bayonet connection, magnets, ball detent).

With continued reference to FIGS. 13-15, the second end 182b defines a fourth diameter D4. As shown in FIG. 13, the second end 182b includes a head 184 that tapers outward from a middle surface 185 of the second connector 180. The middle surface 185 is disposed between the threads 183 and the head 184 and defines a fifth diameter D5, which is smaller than the fourth diameter D4. The diameter D5 is approximately the same magnitude as the diameter D. In the illustrated construction, the fourth diameter D4 is the same magnitude as the first diameter D1. In the illustrated construction, the fifth diameter D5 is configured to match the diameter D of the pylon 104. As shown in FIG. 14, the second end 182b includes apertures 186 configured to align with the apertures 154 of the base 106 (e.g., platform 150 of the base 106. In other words, the apertures 186 are disposed at common radial locations and circumferential locations relative to the longitudinal axis A1 such that apertures 154, 186 align. In the illustrated construction, the apertures 186 extend through the head 184 (e.g., the apertures 186 are through holes). In the illustrated construction, the apertures 186 are equally spaced about the longitudinal axis A1. Specifically, the apertures 186 are spaced apart by 90 degrees about the longitudinal axis A1. The modular prosthesis 100 includes fasteners 187 that couple the pylon 104 to the base 106. More particularly, the fasteners 187 couple the second connector 180, which is coupled to the pylon 104, to the base 106. The fasteners 187 extend through the apertures 154, 186. In some constructions, the modular prosthesis 100 includes a locking element (e.g., a nut) to lock the fasteners 187 to the apertures 154, 186 such that the pylon 104 is coupled to the base 106. In other constructions, the fasteners 187 are self-locking such that the pylon 104 is coupled to the base 106. In the illustrated construction, the fasteners 187 are socket head screws M5×0.8P×20 mm. The pylon 104 is coupled to the base 106 such that that the second end 182b is flush against the top face 153 (FIG. 1). That is, the base 106 and the second connector 180 do not axially overlap along the axis A1.

In some constructions, although not illustrated, the first and second locking mechanisms 108a, 108b include different constructions than the first connector 160 and the second connector 180, respectively. For instance, in some construction, the first locking mechanism 108a utilizes a construction similar to the second connector 180 and the second locking mechanism 108b utilizes a construction similar to the first connector 160. In other constructions, the first and second locking mechanisms 108a, 108b include the same type of connector. For instance, in some constructions, the first locking mechanism 108a utilizes the first connector 160 and the second locking mechanism 108b utilizes the first connector 160. In other constructions, the first and second locking mechanisms 108a, 108b utilize one or more levers, pins, pegs, hook and loop fasteners, screws, and the like to securely couple the prosthetic components. In other examples, the lock mechanism may be a twist-snap lock, a bayonet mount, and the like.

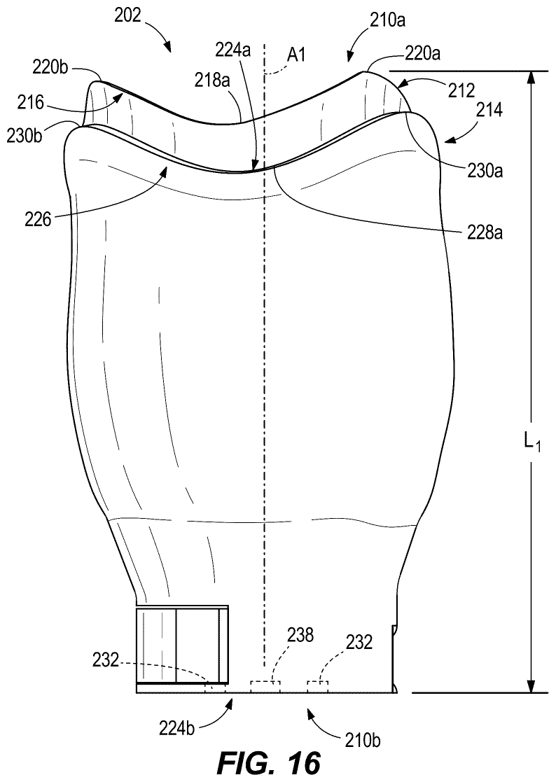

FIG. 16 illustrates another embodiment of a socket assembly 202 that is compatible with the modular prosthesis 100. In other words, the socket assembly 102 is interchangeable with the socket assembly 202. Many features of the socket assembly 202 are similar to those discussed above with regard to the socket assembly 102. As such, many of these features will not be discussed again below. Features similar to those discussed above will be labeled with a reference number that is a value of one hundred higher than the corresponding feature discussed above with respect to the socket assembly 102.

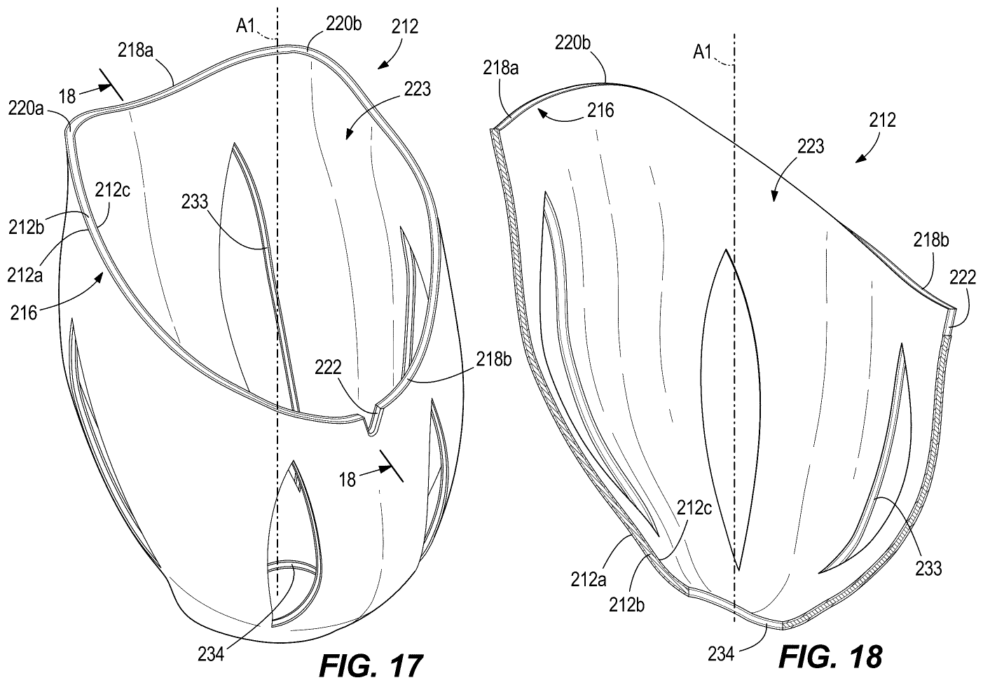

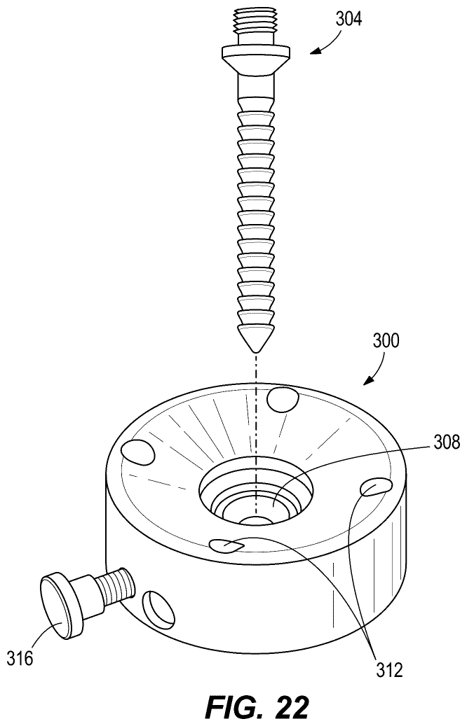

FIGS. 17 and 18 illustrate the inner socket 212. The inner socket 212 includes apertures 233. In the illustrated construction, the inner socket 212 includes an outer layer 212a, a middle layer 212b, and an inner layer 212c. The middle layer 212b is the primary structural component of the inner socket 112 and is constructed of a polymer. Specifically, the middle layer 212b is 3-D printed from a polymer (e.g., PLA, PETG, PLA-CF). The outer layer 212a is disposed between the middle layer 212b and the outer socket 214 and is constructed of a fabric (e.g., Rayon/Nylon/Spandex blend). The outer layer 212a is configured to reduce friction between the middle layer 212b of the inner socket 212 and the outer socket 214. The inner layer 212c is constructed of a heat moldable material (e.g., EVA foam, plastazote) that attaches to the middle layer 212b. The inner layer 212c is conformable to the shape of the user's residual limb. In some constructions, the inner layer 212c is wrapped with fabric to provide additional comfort. The layers 212a-212c each include an aperture 234 configured to receive a pin disposed on the user's residual limb. The shuttle lock mechanism that receives the pin will be discussed further with respect to FIG. 22.

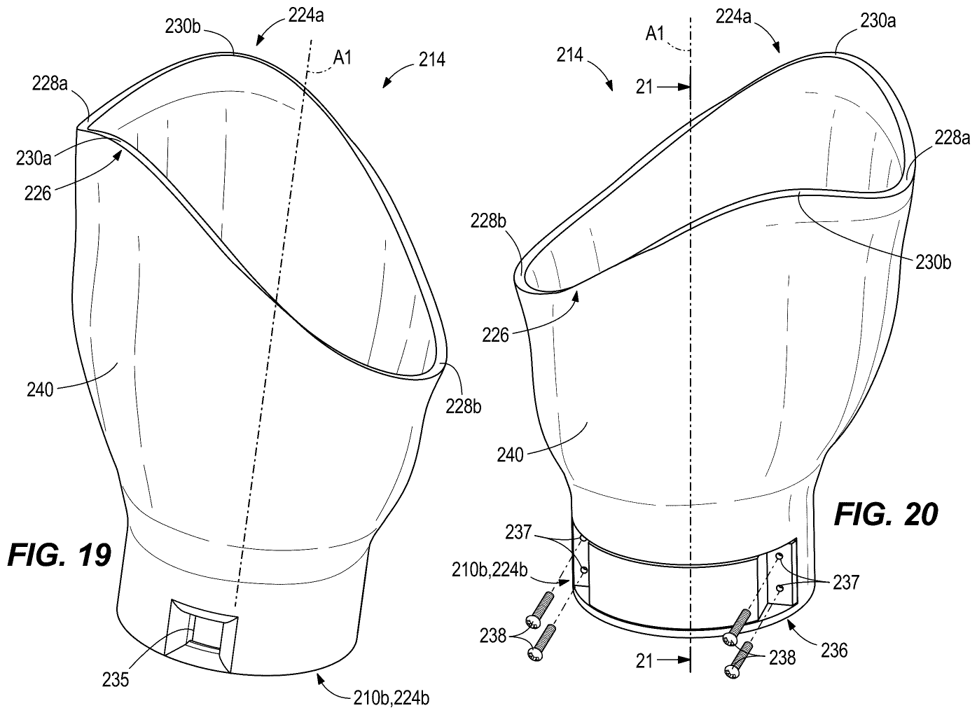

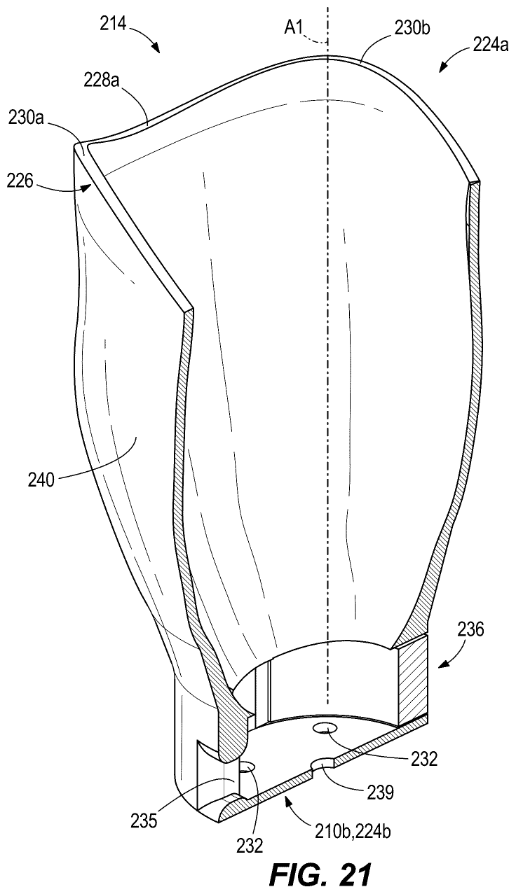

FIGS. 19-21 illustrate the outer socket 214 having a slot 235 and a removable portion 236. The removable portion 236 includes apertures 237 configured to receive fasteners 238 that couple the removable portion 236 to the body of the outer socket 214. In the illustrated construction, the fasteners 238 are pan head sheet metal screws, 6×⅜. The removable portion 236 is selectively removable to the body of the outer socket 214. The outer socket 214 includes a central aperture 239. The body of the outer socket 214 is constructed from a polymer (e.g., PLA, PETG, PLA-CF). The body of the outer socket 214 includes an outer coating 240. The outer coating 240 is a copolymer (e.g., thermacryl).

FIG. 22 illustrates a shuttle lock mechanism 300 that couples the user's residual limb to the modular prosthesis 100. Specifically the shuttle lock mechanism 300 receives a pin 304 disclosed on the user's residual limb. In the illustrated construction, the pin 304 is a notched pin. However, in other constructions, the pin 304 is smooth (i.e., no notches). The shuttle lock mechanism 300 includes a central aperture 308 that revies the pin 304 and includes apertures 312 disposed around the periphery of the central aperture 308. In the illustrated construction, the apertures 312 align with the apertures 232 of the outer socket 214 and the apertures 166 of the second end 162b of the pylon 104. In other words, the apertures 232 are disposed at common radial locations and circumferential locations relative to the longitudinal axis A1 such that apertures 166, 232, and 312 align. In the illustrated construction, the apertures 312 extend through the shuttle lock mechanism 300 (e.g., the apertures 312 are through holes). In the illustrated construction, the apertures 312 are equally spaced about the longitudinal axis A1. Specifically, the apertures 312 are spaced apart by 90 degrees about the longitudinal axis A1. The fasteners 167 extend through the 166, 232, and 312. The shuttle lock mechanism 300 includes a lock 316 extending radially outward from the body of the shuttle lock mechanism 300.

To attach the shuttle lock mechanism 300, the removable portion 236 of the outer socket 214 is removed and the shuttle lock mechanism 300 is inserted into the outer socket 214. That is, the second end 224b of the outer socket 214 defines a cavity for receiving the shuttle lock mechanism 300. The lock 316 is configured to be received by the slot 235 such that the lock 316 is accessible. The fasteners 167 extend through the apertures 312 to couple the shuttle lock mechanism 300 to the outer socket 214.

To couple the user's residual limb to the modular prosthesis 100, the pin 304 disposed on the user's residual limb extends through the aperture 234 in the inner socket 212, the central aperture 239 on the outer socket 214, and into the central aperture 308 of the shuttle lock mechanism 300. Since the shuttle lock mechanism 300 is secured to the outer socket 214 and the first connector 160, the user's residual limb is effectively coupled to the modular prosthesis 100. Depending on the length of the pin 304, the pin may extend through the aperture 168 of the first connector 160. The socket assembly 102 includes the same connection of coupling the user's residual limb to the modular prosthesis 100 as the socket assembly 202.

FIG. 23 illustrates the modular prosthesis 100 with the socket assembly 202. The socket assembly 202 is fitted with a heat source (e.g., microwave, hot water, etc.). Specifically, the inner socket 212 (e.g., the inner layer 212c) is heated up and the user's residual limb is inserted into the inner socket 212. Since the inner socket 212 is formed from a heat moldable material, the user may adjust the inner socket 212 to fit to the shape of the residual limb. Once fitted properly, the user can couple the pin 304 to the shuttle lock mechanism 300. The user then couples the first connector 160 to the second socket end 110b, 210b by tightening the fasteners 167. The fasteners 167 also engage the apertures 312 of the shuttle lock mechanism 300. Alternatively, the socket assembly 202 and the first connector 160 are a single piece. The user then couples the first pylon end 140a to the first connector 160. The user then couples the second connector 180 to the second pylon end 140b. The user then couples the second connector 180 to the base 106 by tightening the fasteners 187. As deemed necessary, the user may increase or decrease the socket length L1, socket width, pylon length L2, and/or pylon width. For instance, the pylon length L2 of a first pylon is less than the pylon length L2 of a second pylon. When the length of the modular prosthesis 100 needs to be an increased, the first pylon can be replaced with the second pylon, thereby increased the pylon length L2.

FIG. 24 illustrates an embodiment of a pylon cover 400. In some constructions, a pylon cover 400 is selectively attachable to the pylon 104. Benefits of the pylon cover 400 include (1) protecting the pylon 104 from fatigue and wear and (2) being aesthetically similar to a human leg. Specifically, the pylon cover 400 defines a shape that mimics the front and rear leg muscles (e.g., the calf muscles, tibialis muscles, and the like). In some constructions, the pylon cover 400 provides a fuller shape to the lower portion of pants, jeans, and the like which may increase a user's confidence and comfortability with using a prosthesis. In some constructions, the pylon cover 400 encases the pylon 104 and attach to the socket assembly 102, 202 and the base 106. In the illustrated construction, the pylon cover 400 defines a length L3 that is the same magnitude as the second length L2.

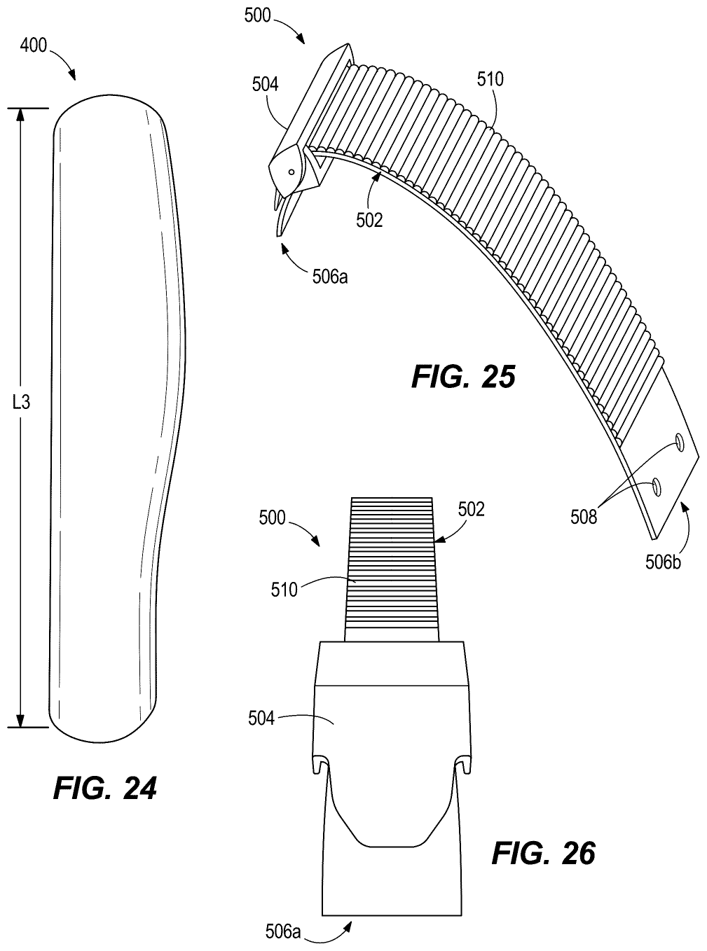

FIGS. 25 and 26 illustrate an optional ratchet strap 500 that is used with the modular prosthesis 100. The ratchet strap 500 is designed to further adjust the fit of the socket assembly 102, 202. In some constructions, the ratchet strap 500 is constructed of polyester, nylon, cotton, and/or a combination of other durable materials. The ratchet strap 500 includes a flexible band 502 and a buckle 504, where the buckle 504 is positioned at a first end 506a of the flexible band 502. Opposite the first end 506a is a second end 506b. The second end 506b includes one or more apertures 508. Moreover, the flexible band 502 includes a plurality of ridges 510 disposed about a length of the flexible band 502 that are configured to provide a more secure fit with the buckle 504. For applying the ratchet strap 500, a user first adorns the modular prosthesis 100. Next, the user wraps the ratchet strap 500 around the socket assembly 102, 202 and feed the flexible band 502 through the buckle 504. The user then continues feeding the flexible band 502 through the buckle 504 until the socket assembly 102, 202 fits snugly around the residual limb. Upon achieving a desirable fit, the user then secures the ratchet strap 500 by engaging the buckle 504 and locking the ratchet strap 500 in place. It would be understood by those of ordinary skill in the art that other means of securing and/or adjusting a fit of the socket assembly 102, 202 may be used instead of the ratchet strap 500. For example, the modular prosthesis 100 may include a hook and loop fastener, a hasp latch, a hook clasp, a toggle clasp, a bailing latch, one or more pings, and the like. The user may adjust a width of the socket assembly 102, 202 by using the ratchet strap 500.

In some constructions, the user is able to adjust the modular prosthesis 100 by using additional tools. For example, the user may use a combination of screws and screwdrivers, bolts and wrenches, and other conventional tools to secure the socket assembly 102, 202, the pylon 104, and the base 106.

In some constructions, although not illustrated, the modular prosthesis 100 is an upper extremity prosthetic. In such embodiments, the modular upper extremity prosthesis includes substantially similar materials. Further, the modular upper extremity prosthesis includes scalable and adjustable components and one or more locking mechanisms (e.g., the first connector 160 and a corresponding threaded connector) to allow for personalization and customization. Yet further, it is to be understood that the teachings described herein applies to prostheses relating to various areas of human anatomy and is not necessarily limited to lower and upper extremities.

In other alternative instances, although not illustrated, the modular prosthesis 100 may not include a base 106. In such instances, the modular prosthesis 100 may include an ankle portion and a foot portion that is selectively attachable to each other and/or other prosthetic components (e.g., the pylon 104). For example, the ankle portion may couple to the second pylon end 140b and the foot portion may couple to the ankle portion.

In some constructions, although not illustrated, the base 106 may include a foot-cover that is designed to be substantially aesthetically similar to a human foot. For example, the foot-cover is configured to engage, interact, or couple to at least a portion of the base 106. Further, the foot-cover may couple to the pylon 104. In some constructions, the modular prosthesis 100 may include one or more prosthetic component covers to provide aesthetics substantially similar to the respective human anatomy.

It will be appreciated by those skilled in the art that scalability of the modular prosthesis 100 allows for the user to gain independence. That is, because the user is able to adjust aspects of the modular prosthesis 100 (for example, widths and lengths), the user would no longer be limited to prosthetist availability to improve the size and fit of their prosthetic device. Additionally, because the modular prosthesis 100 is manipulated to accommodate different stages of life, the user is able to keep and utilize the modular prosthesis 100 for a longer duration than current solutions. Moreover, because the modular prosthesis 100 is selectively constructed and deconstructed, the user may attach and detach the modular prosthesis 100 as desired and travel with a deconstructed configuration of the modular prosthesis 100 without having to worry about a cumbersome re-assembly.

An innovative prosthetic (e.g., a modular prosthetic), paired with telehealth software, eliminates much of the challenges in the current prosthetic fitting and repair process for both the patient and the prosthetist.

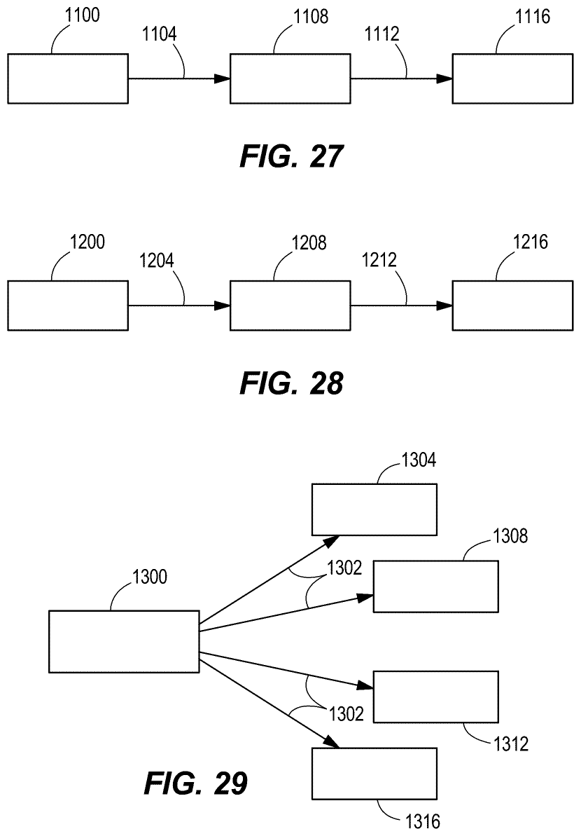

FIG. 27 illustrates a typical procedure for maintenance repair of a prosthetic (not shown). First, a patient must visit a physician 1100 and receive a prescription 1104 for visiting a prosthetist 1108. After visiting the prosthetist 1108, the prosthetist 1108 will submit a claim with insurance and await approval from the insurer. After an approved claim 1112, the prosthetist 1108 will make a repair 1116 to the patient's prosthetic (e.g., if the patient wants to have insurance cover the cost). However, depending on the terms of the patient's insurance policy, the insurance company may or may not cover the cost of repair in whole or in part. As such, patients may forego the repair based on circumstances (e.g., a financial situation), which may result in the patient skipping the maintenance repair. If the maintenance repair is not completed, the patient will have to make do with an ill-fitting or poorly functioning prosthetic.

FIG. 28 illustrates the procedure of an initial fitting of a prosthetic (e.g., a modular prosthetic) for a patient. To obtain a prosthetic, the patient's journey begins by visiting a physician 1200 and receiving a prescription 1204 for visiting a prosthetist 1208. After visiting the prosthetist 1208, the prosthetist 1208 will submit a claim with insurance and await approval from the insurer. After an approved claim 1212, the prosthetist 1208 will complete a fitting 1216 of the prosthetic with the patient. In some constructions, insurance may not approve the claim, and the patient may pay out of pocket for the prosthetic.

FIG. 29 illustrates a prosthetic maintenance program. Following the initial fitting illustrated in FIG. 28, the maintenance of the prosthetic is different compared to the typical prosthetic maintenance illustrated in FIG. 27 due to the prosthetic maintenance program. After being fitted for a prosthetic, a patient can elect to begin paying a fee for a membership or subscription 1300 to the program provider to spread out the cost of maintenance over the life of the unit. The subscription 1300 gives the patient access to additional options that are not available for the typical prosthetic maintenance (FIG. 27).

First, the subscription 1300 enables access to a platform 1302 to book appointments. In some constructions, the platform 1302 may be an internet portal such that appointments may be booked quickly online.

Next, the subscription 1300 enables access to schedule appointments with a prosthetist 1304 to get support for the prosthetic. The appointments with the prosthetist 1304 may be virtual or in-person. Compared with the typical prosthetic maintenance illustrated in FIG. 27, the visit to the physician is not necessary because the patient may directly book with the prosthetist 1304 without a prescription. As such, the patient saves money and time by avoiding a visit to the physician.

Also, the subscription 1300 enables access for the patient to order parts 1308 directly to their home. For instance, after meeting with the prosthetist 1304, the prosthetist 1304 may recommend ordering parts for certain maintenance repairs. Also, the subscription 1300 enables access for the patient to order the parts on their own, without an order from the prosthetist 1304. The subscription 1300 allows the patient to pay for parts 1308 out-of-pocket via the fee for the subscription 1300. In some embodiments, the cost of the parts 1308 are covered by the fee for the subscription 1300. That is, the cost for the parts 1308 needed by the patient are covered by the fee for the subscription 1300. In some embodiments, the cost of the parts 1308 are not covered by the fee for the subscription 1300 and would be an additional cost to the patient.

Additionally, the subscription 1300 enables access to an in-home repair network 1312. The in-home repair network 1312 enables access to prosthetists that can help with repairs of the prosthetic. For instance, after receiving parts 1308, a patient may utilize the in-home repair network 1312 to receive guidance on how to implement the parts 1308 with the prosthetic. The prosthetists for the in-home repair network 1312 may help in-person or virtually. Additionally, the in-home repair network 1312 may include access to certified nursing assistants (CNAs) and emergency medical technicians (EMTs). The in-home repair network 1312 differs from the existing customer journey in that it removes the friction of unknown pricing and long appointment wait times. If the patient opts to visit the prosthetist, the same current model insurance approval process will still apply, as shown in FIG. 27.

Optionally, the subscription 1300 enables access to in-person assistance 1316 from a medical professional such as an EMT or CNA. The EMT/CNAs would work part time for the service provider on a contract basis. They would be given the option to select from an available pool of working hours in a specific location, working anywhere from 5-20 hours per week. The patient may select an appointment time using the platform 1302. For instance, certain time slots will be marked as having a CNA/EMT availability and the patient may select one of these slots and opt whether the appoint is in-person or virtual.

In the prosthetic maintenance program, prosthetics are sold directly to prosthetist offices and the prosthetist then sells the prosthetic to patients and receives reimbursement through insurance. With respect to the subscription 1300, the patient, or end-user, pays the fee for the subscription 1300 to the service provider. This allows the patient to spread the cost of repair out over time, making repairs affordable.

From the service provider's end of the transaction, the service provider will make money both from selling the original model to the prosthetist (e.g., business-to-business), and then from the patient's subscription (e.g., business-to-consumer). The prosthetist who fits the prosthetic makes money from re-selling the service provider's prosthetic to the patient and handles any transaction with the patient's insurance regarding initial and any subsequent in-office repair costs. The reason for initially utilizing the local prosthetist is that, for the initial fitting, certain measurements need to be done in person. Once these measurements are done, the patient can utilize these same measurements for subsequent repairs. However, additional measurements may need to be taken (e.g., subsequent measurements). For instance, weight fluctuations may warrant subsequent measurements depending on the fit of the prosthetic.

With the subscription 1300, the patient may schedule a telehealth appointment whenever they wish to make their own adjustments or repairs, instead of needing to wait for insurance approval. On average, the first socket of the prosthetic lasts about 6 months. That is, the socket may loosen due to changes in their residual limb. The patient can leverage the subscription 1300 to schedule a telehealth appointment with the prosthetist who can walk them through how to alter the shape of their socket's heat moldable inner layer and utilize the ratchet straps on the outer layer to achieve a tighter fit. With the subscription 1300, patients can schedule online telehealth appointments to make these types of adjustments throughout the lifespan of their prosthetic.

The subscription 1300 appeals to both prosthetists and patients. Patients can repair or adjust their prosthetic quickly, without the delay or concern of obtaining insurance approval. Since repairs can be done in the patient's home using common, ordinary tools, the prosthetic is easy to customize and maintain. As such, the subscription 1300 eliminates pricing uncertainties and appointment delays and prioritizes patient convenience and comfort thereby ensuring a precise and comfortable lifetime fit at an affordable cost.

In the typical repair process (FIG. 27), most insurance carriers follow Medicare DMEOP (Durable Medical Equipment, Prosthetics, Orthotics) rules. Prosthetists are not reimbursed for time, but rather for parts sold. As a result, prosthetists are incentivized to sell prosthetics rather than repair the prosthetics (i.e., without buying additional parts) because prosthetists may not be compensated if no additional parts of the prosthetics are ordered. The subscription 1300 empowers patients to perform DIY repairs or access a network of in-home repair and modification providers for a small additional fee.

The service provider may include a relief fund for patients that is accessible to patients with the subscription 1300 that need financial support. For instance, the relief fund may include an application such that patients with the subscription 1300 can apply for financial support. After a patient with the subscription 1300 is approved for financial support, the patient may receive financial support from the relief fund.

Although the disclosure has been described in detail with reference to certain preferred embodiments, variations and modifications exist within the scope and spirit of one or more independent aspects of the disclosure as described.

Various features of the disclosure are set forth in the following claims.

What is claimed is:

1. A modular prosthesis comprising:
a socket assembly including an outer socket and an inner socket coupled to the outer socket;
a base configured to contact a ground surface;
a pylon disposed between the socket assembly and the base, the pylon defining a longitudinal axis, the pylon having a first end and a second end opposite from the first end along the longitudinal axis, the first end coupled to the socket assembly and the second end coupled to the base;
a first locking mechanism disposed between the socket assembly and the pylon; and
a second locking mechanism disposed between the pylon and the base.

2. The modular prosthesis of claim 1, wherein the first locking mechanism includes a first connector having a first end and a second end opposite the first end, and
wherein the first end of the first connector is coupled to the first end of the pylon and the second end of the first connector is coupled to the socket assembly.

3. The modular prosthesis of claim 2, wherein the first end of the first connector includes a receptacle configured to receive the first end of the pylon.

4. The modular prosthesis of claim 2, wherein the second end of the first connector includes apertures, wherein a bottom of the outer socket includes apertures, and wherein the apertures of the first connector radially and circumferentially align with the apertures of the bottom of the outer socket relative to the longitudinal axis such that fasteners couple the first connector to the socket assembly.

5. The modular prosthesis of claim 1, wherein the second locking mechanism includes a second connector having a first end and a second end opposite the first end, and wherein the first end of the second connector is coupled to the second end of the pylon and the second end of the second connector is coupled to the base.

6. The modular prosthesis of claim 5, wherein the second end of the pylon includes a threaded receptacle configured to receive the first end of the second connector having threads.

7. The modular prosthesis of claim 5, wherein the base includes a platform, a blade extending from the platform, and a heel coupled to the blade.

8. The modular prosthesis of claim 7, wherein the platform and the blade are constructed from a single part, wherein the heel is formed separately from the platform and the blade, and wherein a fastener is configured to couple the blade and the heel.

9. The modular prosthesis of claim 7, wherein the heel defines a U-shape having ends, and wherein each of the ends are configured to engage a surface.

10. The modular prosthesis of claim 7, wherein the second end of the second connector includes apertures, wherein the platform of the base includes apertures, and wherein the apertures of the second connector radially and circumferentially align with the apertures of the platform relative to the longitudinal axis such that fasteners couple the second connector to the base.

11. The modular prosthesis of claim 10, wherein the apertures of the second end of the second connector and the apertures of the platform are through holes having threads.

12. The modular prosthesis of claim 1, further including a shuttle lock mechanism and wherein the outer socket includes a slot configured to receive a portion of the shuttle lock mechanism.

13. The modular prosthesis of claim 12, wherein the shuttle lock mechanism includes an aperture configured to receive a pin disposed on a residual limb, and wherein the outer socket includes a second end defining a central aperture configured to axially align with the aperture of the shuttle lock mechanism.

14. The modular prosthesis of claim 1, wherein the inner socket includes an outer layer, a middle layer, and an inner layer, wherein the outer layer is constructed of a fabric and is disposed between the middle layer and the outer socket, wherein the middle layer is constructed of a polymer, and wherein the inner layer is constructed of a heat moldable material configured to conform to a shape of a residual limb.

15. The modular prosthesis of claim 1, wherein the inner socket and the outer socket each include an upper periphery that defines at least one peak and at least one dip, wherein the at least one dip is disposed axially closer to pylon than the at least one peak relative to the longitudinal axis.

16. The modular prosthesis of claim 1, wherein the pylon defines a diameter, wherein a portion of the first locking mechanism defines a diameter, wherein a magnitude of the diameters of the pylon and the first locking mechanism are approximately equal.

17. The modular prosthesis of claim 1, further comprising a pylon cover configured to be selectively coupled to the pylon, wherein the pylon defines a length along the longitudinal axis, and wherein the pylon cover defines a length along the longitudinal axis having a similar magnitude to the length of the pylon.

18. A method of fitting and assembling a modular prosthesis, the method comprising:
heating an inner socket with a heat source;
conforming a residual limb to the inner socket;
coupling, with a shuttle lock, the inner socket to an outer socket;
coupling, with fasteners, a first connector to the outer socket;
coupling, with a threaded connection, a first end of a first pylon to the first connector of a first locking mechanism;
coupling, with a threaded connection, a second end of the first pylon to a second connector of a second locking mechanism; and
coupling, with fasteners, the second connector to a base.

19. The method of claim 18, further comprising
removing the first pylon from the first and the second connectors;
coupling a second pylon to the first and second connectors, the second pylon having a different length than the first pylon.
